(19) United States
(12) Patent Application Publication
(10) Pub. No.: US 2025/0232412 A1
(43) Pub. Date: Jul. 17, 2025

(54) INTRAVASCULAR IMAGE NOISE ARTIFACT REDUCTION

(71) Applicant: Boston Scientific Scimed Inc., Maple Grove, MN (US)

(72) Inventors: Hatice Cinar Akakin, Eden Prairie, MN (US); Corydon Carlson, Stillwater, MN (US); Kevin Bloms, Minneapolis, MN (US); Wenguang Li, Los Gatos, CA (US)

(73) Assignee: Boston Scientific Scimed Inc., Maple Grove, MN (US)

(21) Appl. No.: 19/016,342

(22) Filed: Jan. 10, 2025

Related U.S. Application Data
(60) Provisional application No. 63/620,570, filed on Jan. 12, 2024.

(51) Int. Cl. G06T 5/60; A61B 8/00; A61B 8/08; A61B 8/12; G06T 5/70

(52) U.S. Cl. CPC G06T 5/60; A61B 8/12; A61B 8/5269; G06T 5/70; A61B 8/0891; G06T 2207/10101; G06T 2207/10132; G06T 2207/20081; G06T 2207/20084; G06T 2207/30101

(57) ABSTRACT

The present disclosure provides apparatus and methods to train ML models to infer intravascular images with noisy artifacts removed from intravascular images comprising the noisy artifacts. Further, the disclosure provides a computing system that can be part of an intravascular image capture device configured to infer cleaned images from raw images captured by the intravascular imaging device.

### CROSS-REFERENCE TO RELATED APPLICATIONS

This application claims the benefit of U.S. Provisional Application No. 63/620,570, filed Jan. 12, 2024, the disclosure of which is incorporated herein by reference in its entirety.

### TECHNICAL FIELD

The present disclosure pertains to reducing noise artifacts and improving image quality in intravascular images, such as intravascular ultrasound (IVUS) images or intravascular optical coherence tomography (OCT) images.

### BACKGROUND

Intravascular imaging, such as IVUS or OCT, is widely used in interventional cardiology. For example, such images are used to determine the severity of a stenosis in a blood vessel. These intravascular images are also often relied on to determine characteristics of the blood vessel, such as, for example, lumen and vessel borders, plaque burden, locations of side branches, etc.

Many intravascular image modalities are subject to noise. High definition IVUS images are prone to a several different types of noise artifacts. Such noise artifacts can affect the deterministic quality of the images. As such, there is a need to reduce the artifacts resulting from noise in intravascular images.

### BRIEF SUMMARY

The present disclosure provides to reduce and/or remove artifacts resulting from noise in intravascular images. In general, the present disclosure provides to train machine learning (ML) models to generate a cleaned image from a noisy image. Such ML models can be implemented in a commercial intravascular imaging system and configured to take raw (e.g., unfiltered, unprocessed, or the like) images captured from an intravascular image capture device and generate a filtered or cleaned output image where the filtered or cleaned output image has reduced artifacts from noise. In some embodiments, the present disclosure provides example training data sets and algorithms to train such ML models.

The present disclosure provides an advantage over conventional filtering techniques. For example, current filtering or noise reduction algorithms are overly computationally burdensome and/or too time consuming to be implemented commercially. Current methods are also prone to removing valid signals since the bandwidth of noise and signal often overlaps. As the resolution of intravascular imaging (e.g., high-definition IVUS, etc.) increases, the susceptibility to noise also increases. Thereby necessitating a need for the present disclosure. Further, as the intravascular images are often used for deterministic purposes (e.g., border and lumen assessment, stent location identification, or the like) consistency in image quality is needed for such deterministic algorithms to be commercially useful.

In some embodiments, the disclosure can be implemented as a method for removing noisy artifacts from a series of intravascular images. The method can comprise receiving, at a computing device from an intravascular imaging device, a plurality of images associated with a vessel of the patient, the plurality of images comprising multidimensional and multivariate images; and inferring, by the computing device using a machine learning (ML) model, a plurality of cleaned images corresponding to the plurality of images, wherein the plurality of images comprise one or more noisy artifacts and wherein at least one of the one or more noisy artifacts is removed from the plurality of cleaned images.

With further embodiments, the method can comprise generating, by the computing device, a graphical information element comprising an indication of the plurality of cleaned images; and causing, by the computing device, the graphical information element to be displayed on a display coupled to the computing device.

With further embodiments of the method, the plurality of images are intravascular ultrasound (IVUS) images.

With further embodiments of the method, the ML model comprises a plurality of ML models and wherein inferring the plurality of cleaned images comprises generating an inference from the plurality of ML models in serial.

With further embodiments of the method, the ML model comprises a first ML model and a second ML model and wherein inferring the plurality of cleaned images can comprise inferring, by the computing device using the first ML model with the plurality of images as input, a plurality of intermediate cleaned images corresponding to the plurality of images; and inferring, by the computing device using the second ML model with the plurality of intermediate cleaned images as input, the plurality of cleaned images corresponding to the plurality of images.

With further embodiments of the method, the ML model comprises a neural network (NN) or a convoluted neural network (CNN) architecture.

With further embodiments of the method, the ML model is trained using a supervised training algorithm with a training data set that comprises a plurality of images comprising one or more noisy artifacts and, for each of the plurality of images, a ground truth image that does not comprise the one or more noisy artifacts.

With further embodiments of the method, the ML model is trained using an unsupervised training algorithm with a training data set that comprises a plurality of images comprising one or more noisy artifacts and wherein the network is configured to receive at least two or more of the plurality of images as input and generate a cleaned image corresponding to one of the at least two or more of the plurality of images, wherein the cleaned image does not comprise the one or more noisy artifacts.

With further embodiments of the method, the ML model is trained using a Diffusion Network training algorithm iteratively optimizing the network parameters.

With further embodiments of the method, the ML model is trained using a generative adversarial network (GAN) training algorithm comprising a discriminator network.

With further embodiments of the method, the GAN training algorithm comprises an auxiliary task network.

With further embodiments of the method, the one or more noisy artifacts correspond to blood speckle noise.

With further embodiments of the method, the one or more noisy artifacts correspond to electromagnetic interference or radio frequency interference.

In some embodiments, the disclosure can be implemented as a computing device to couple to an intravascular imaging device, the computing device comprising a processor and memory, the memory comprising instructions that when executed by the processor cause the computing device to implement any of the methods described herein.

In some embodiments, the disclosure can be implemented as one or more computer readable storage devices comprising instructions, which when executed by a processor of a computing device configured to couple to an intravascular imaging device, cause the computing device to implement any of the methods described herein.

In some embodiments, the disclosure can be implemented as a computing device to couple to an intravascular imaging device. The computing device can comprise a processor; an interconnect coupled to the processor, the interconnect configured to couple to an intravascular imaging device; and a memory device, the memory device comprising instructions that when executed by the processor cause the processor to receive, from the intravascular imaging device, a plurality of images associated with a vessel of a patient, the plurality of images comprising multidimensional and multivariate images; and infer, using a machine learning (ML) model, a plurality of cleaned images corresponding to the plurality of images, wherein the plurality of images comprises one or more noisy artifacts and wherein at least one of the one or more noisy artifacts is removed from the plurality of cleaned images.

With further embodiments of the computing device, the instructions when executed further cause the processor to generate a graphical information element comprising an indication of the plurality of cleaned images; and cause the graphical information element to be displayed on a display coupled to the computing device.

With further embodiments of the computing device, the plurality of images are intravascular ultrasound (IVUS) images.

With further embodiments of the computing device, the ML model comprises a plurality of ML models and wherein inferring the plurality of cleaned images comprises generating an inference from the plurality of ML models in serial.

With further embodiments of the computing device, the ML model comprises a first ML model and a second ML model and wherein inferring the plurality of cleaned images comprises inferring, by the computing device using the first ML model with the plurality of images as input, a plurality of intermediate cleaned images corresponding to the plurality of images; and inferring, by the computing device using the second ML model with the plurality of intermediate cleaned images as input, the plurality of cleaned images corresponding to the plurality of images.

With further embodiments of the computing device, the ML model comprises a neural network (NN) or a convoluted neural network (CNN) architecture.

With further embodiments of the computing device, the ML model is trained using a supervised training algorithm with a training data set that comprises a plurality of images comprising one or more noisy artifacts and, for each of the plurality of images, a ground truth image that does not comprise the one or more noisy artifacts.

With further embodiments of the computing device, the ML model is trained using an unsupervised training algorithm with a training data set that comprises a plurality of images comprising one or more noisy artifacts and wherein the network is configured to receive at least two or more of the plurality of images as input and generate a cleaned image corresponding to a one of the at least two or more of the plurality of images, wherein the cleaned image does not comprise the one or more noisy artifacts.

In some embodiments, the disclosure can be implemented as one or more computer readable storage devices comprising instructions, which when executed by a processor of a computing device configured to couple to an intravascular imaging device, cause the computing device to receive, from the intravascular imaging device, a plurality of images associated with a vessel of a patient, the plurality of images comprising multidimensional and multivariate images; and infer, using a machine learning (ML) model, a plurality of cleaned images corresponding to the plurality of images, wherein the plurality of images comprises one or more noisy artifacts and wherein at least one of the one or more noisy artifacts is removed from the plurality of cleaned images.

With further embodiments of the one or more computer readable storage devices, the plurality of images are intravascular ultrasound (IVUS) images.

With further embodiments of the one or more computer readable storage devices, the one or more noisy artifacts correspond to blood speckle noise, electromagnetic interference, and/or radio frequency interference.

With further embodiments of the one or more computer readable storage devices, the ML model comprises a plurality of ML models and wherein inferring the plurality of cleaned images comprises generating an inference from the plurality of ML models in serial.

With further embodiments of the one or more computer readable storage devices, the ML model comprises a first ML model and a second ML model and wherein inferring the plurality of cleaned images comprises inferring, by the computing device using the first ML model with the plurality of images as input, a plurality of intermediate cleaned images corresponding to the plurality of images; and inferring, by the computing device using the second ML model with the plurality of intermediate cleaned images as input, the plurality of cleaned images corresponding to the plurality of images.

With further embodiments of the one or more computer readable storage devices, the ML model is trained using a Diffusion Network training algorithm iteratively optimizing the network parameters.

With further embodiments of the one or more computer readable storage devices, the ML model is trained using a generative adversarial network (GAN) training algorithm comprising a discriminator network.

With further embodiments of the one or more computer readable storage devices, the GAN training algorithm comprises an auxiliary task network.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates an intravascular treatment system in accordance with an embodiment.

FIG. 2 illustrates a logic flow for reducing noise artifacts in an intravascular image or images in accordance with an embodiment.

FIG. 3A illustrates an example intravascular image comprising noisy artifacts.

FIG. 3B illustrates the example intravascular image of FIG. 3A with ones of the noisy artifacts removed in accordance with an embodiment.

FIG. 4A, FIG. 4B, and FIG. 4C illustrate example intravascular images comprising noise artifacts.

FIG. 5 illustrates an exemplary artificial intelligence/machine learning (AI/ML) training system suitable for use with exemplary embodiments.

FIG. 6 illustrates an example ML network architecture, in accordance with an embodiment.

FIG. 7 illustrates another example ML network architecture, in accordance with an embodiment.

FIG. 8 illustrates yet another example ML network architecture, in accordance with an embodiment.

FIG. 9 illustrates a computer-readable storage medium in accordance with one embodiment.

FIG. 10A illustrates an intravascular treatment system in accordance with an embodiment.

FIG. 10B and FIG. 10C illustrate portions of the intravascular treatment system of FIG. 10A.

FIG. 11 illustrates a diagrammatic representation of a machine in the form of a computer system within which a set of instructions may be executed for causing the machine to perform any one or more of the methodologies discussed herein, according to an example embodiment.

### DETAILED DESCRIPTION

As introduced above, in an exemplary embodiment, a system is provided that is configured to remove noise artifacts from intravascular images (e.g., intravascular ultrasound (IVUS), or the like) by inference from machine learning (ML) models.

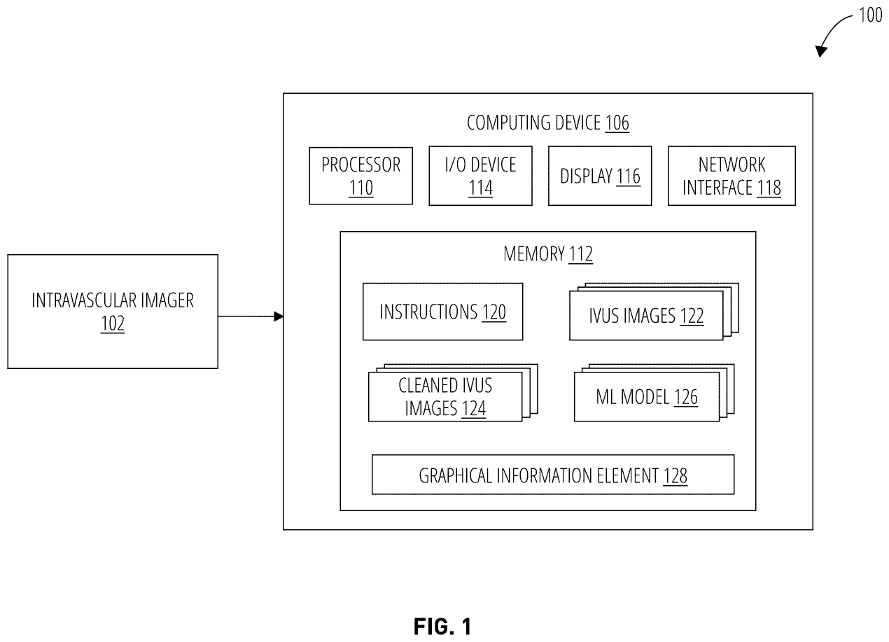

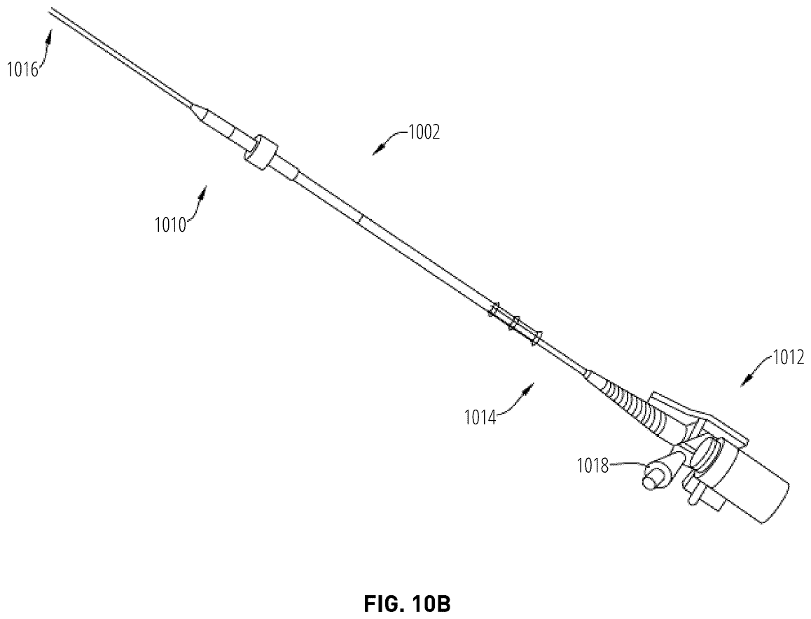

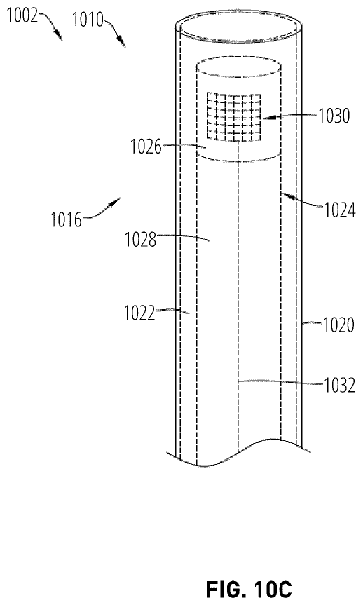

FIG. 1 illustrates a noise artifact removal system 100, in accordance with an embodiment of the present disclosure. In general, noise artifact removal system 100 is a system for removing noise artifacts from intravascular images. To that end, noise artifact removal system 100 includes an intravascular imager 102, and a computing device 106. Intravascular imager 102 can any of a variety of intravascular imagers (e.g., IVUS, OCT, OCE, or the like). In a specific example, the intravascular imager 102 can be the intravascular treatment system 1000 described with reference to FIG. 10A below. Although noise artifact removal system 100 can be implemented to operate on any of a variety of intravascular image modalities, the present disclosure often uses the IVUS modality for examples.

Noise artifact removal system 100 can be provisioned with ML models, which are trained to infer cleaned images, or rather, images with noise artifacts removed. Noise artifact removal system 100 provides a significant advantage over conventional systems. For example, algorithms that operate on IVUS images (e.g., lumen and border detection algorithms, side branch detection algorithms, stent detection algorithms, etc.) can benefit from those noise reduced images.

Computing device 106 can be any of a variety of computing devices. In some embodiments, computing device 106 can be incorporated into and/or implemented by a console of intravascular imager 102. With some embodiments, computing device 106 can be a workstation or server communicatively coupled to intravascular imager 102. With still other embodiments, computing device 106 can be provided by a cloud based computing device, such as, by a computing as a service system accessibly over a network (e.g., the Internet, an intranet, a wide area network, or the like). Computing device 106 can include a processor 110, a storage device or memory 112, an input and/or output (I/O) device 114, a display 116, and a network interface 118.

The processor 110 may include circuitry or processor logic, such as, for example, any of a variety of commercial processors. In some examples, processor 110 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 110 may include graphics processing portions and may include dedicated memory, multiple-threaded processing and/or some other parallel processing capability. In some examples, the processor 110 may be an application specific integrated circuit (ASIC) or a field programmable integrated circuit (FPGA).

The memory 112 may include logic, a portion of which includes arrays of integrated circuits, forming non-volatile memory to persistently store data or a combination of non-volatile memory and volatile memory. It is to be appreciated, that the memory 112 may be based on any of a variety of technologies. In particular, the arrays of integrated circuits included in memory 112 may be arranged to form one or more types of memory, such as, for example, dynamic random access memory (DRAM), NAND memory, NOR memory, or the like.

I/O devices 114 can be any of a variety of devices to receive input and/or provide output. For example, I/O devices 114 can include, a keyboard, a mouse, a joystick, a foot pedal, a haptic feedback device, an LED, or the like.

Display 116 can be a conventional display or a touch-enabled display. Further, display 116 can utilize a variety of display technologies, such as, liquid crystal display (LCD), light emitting diode (LED), or organic light emitting diode (OLED), or the like.

Network interface 118 can include logic and/or features to support a communication interface. For example, network interface 118 may include one or more interfaces that operate according to various communication protocols or standards to communicate over direct or network communication links. Direct communications may occur via use of communication protocols or standards described in one or more industry standards (including progenies and variants). For example, network interface 118 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 118 can include logic and/or features to enable communication over a variety of wired or wireless network standards (e.g., 802.11 communication standards). For example, network interface 118 may be arranged to support wired communication protocols or standards, such as, Ethernet, or the like. As another example, network interface 118 may be arranged to support wireless communication protocols or standards, such as, for example, Wi-Fi, Bluetooth, ZigBee, LTE, 5G, or the like.

Memory 112 can include instructions 120, IVUS images 122, cleaned IVUS images 124, ML model 126, and graphical information element 134. During operation, processor 110 can execute instructions 120 to cause computing device 106 to receive IVUS images 122 from intravascular imager 102. In general, IVUS images 122 are multi-dimensional multivariate images comprising indications of the vessel type, a lesion in the vessel, the lesion type, a stent or stents, the lumen border, the lumen dimensions, the minimum lumen area (MLA), the media border (e.g., a media border for media within the blood vessel), the media dimensions, the calcification angle/arc, the calcification coverage, combinations thereof, and/or the like.

Processor 110 can further execute instructions 120 to cause computing device 106 to infer cleaned IVUS images 124 from IVUS images 122 and ML model 126. With some embodiments, processor 110 can execute instructions 120 to cause computing device 106 to execute ML model 126 with IVUS images 122 as inputs to generate cleaned IVUS images 124. Examples of the network architecture and training of ML model 126 are provided below. However, in general, ML model 126 (e.g., a neural network (NN), a convolutional neural network (CNN), etc.) is trained to generate noise reduced IVUS images from noisy IVUS images. Both noisy and/or clean IVUS images can be collected for a training dataset. Noisy images with different underlying noise sources can be included in the training dataset. Additionally, some clean IVUS images may be used to create synthetically generated noisy images from known noise patterns (e.g., using generative AI methods, or the like) to supplement the quantity of the training dataset.

Processor 110 can further execute instructions 120 to cause computing device 106 to determine graphical information element 128 from cleaned IVUS images 124. For example, processor 110 can execute instructions 120 to generate a graphical element (e.g., for a graphical user interface, or the like) including indications of the IVUS images 122 and/or cleaned IVUS images 124. Further, processor 110 can execute instructions 120 to cause computing device 106 to display the graphical information element 128 on the display 116.

With some embodiments, memory 112 can include multiple ML models 126. For example, a first one of ML models 126 could be trained to remove noise artifacts from blood speckle while a second one of ML models 126 could be trained to remove noise artifacts from electromagnetic interference. In such an example, the IVUS images 122 can be fed into the ML models 126 serially to generate cleaned IVUS images 124. For example, an intermediate image (not shown) can be inferred from IVUS images 122 and the first one of ML models 126 and cleaned IVUS images 124 can be inferred from the intermediate image and the second one of ML models 126. Examples are not limited in this context.

Further, it is noted that IVUS images 122 will include a series of images, such as, several image frames, or the like. Accordingly, ML model 126 can be configured to generate a one of the cleaned IVUS images 124 from a corresponding one of the IVUS images 122, resulting in a series of cleaned images.

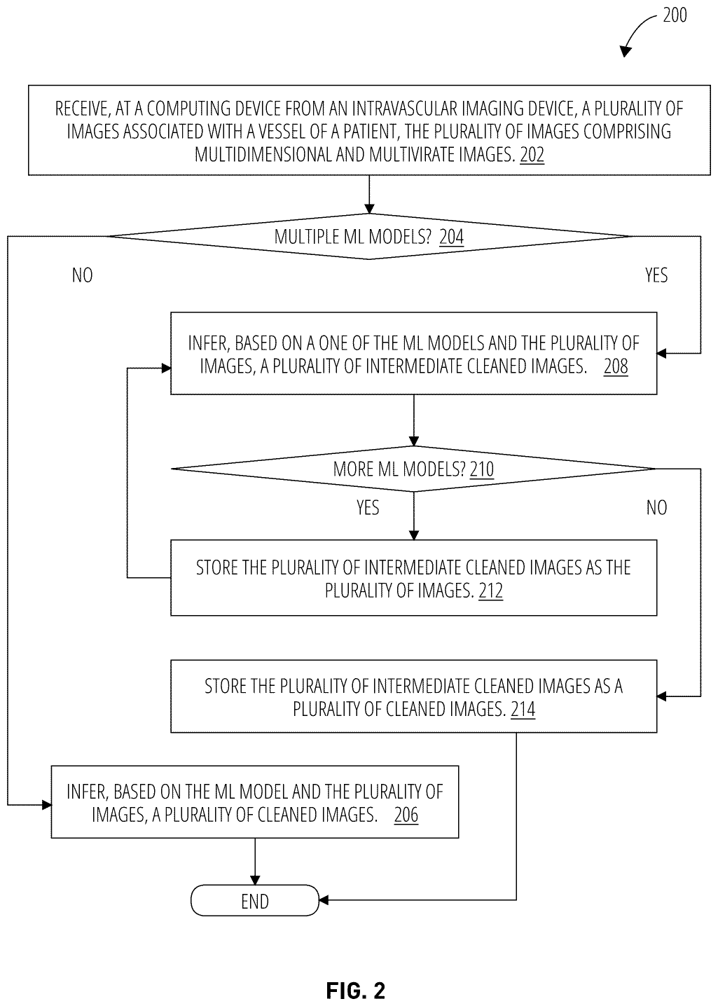

FIG. 2 illustrates routine 200 according to some embodiments of the present disclosure. Routine 200 can be implemented by noise artifact removal system 100 or another computing device as outlined herein remove (or reduce) noise artifacts from IVUS images.

Routine 200 can begin at block 202. At block 202 “receive, at the computing device from an intravascular imaging device, a plurality of images associated with a vessel of a patient, the plurality of images comprising multidimensional and multivirate images” computing device 106 of noise artifact removal system 100 receives IVUS images 122 from intravascular imager 102 where IVUS images 122 are multidimensional and multivirate images of the vessel. For example, processor 110 can execute instructions 120 to receive data including indications of IVUS images 122 from intravascular imager 102 via network interface 118.

Continuing to decision block 204 “multiple ML models?” a determination of whether multiple ML models are provided. For example, processor 110 of computing device 106 can execute instructions 120 to determine whether one of ML models 126 or multiple ones of ML models 126 are provided. As outlined above, with some embodiments, one ML model 126 is provided to remove noise artifacts from IVUS images 122, whereas, in other embodiments, multiple ML models 126 can be provided to remove noise artifacts (e.g., resulting from different types of noise, or the like). From decision block 204, routine 200 can continue to either block 206 or block 208. Routine 200 can continue from decision block 204 to block 206 based on a determination at decision block 204 that a single ML model is provided, whereas routine 200 can continue from decision block 204 to block 208 based on a determination at decision block 204 that multiple ML models are provided.

At block 206 “infer, based on the ML model and the plurality of images, a plurality of cleaned images” a plurality of cleaned images can be inferred from the plurality of images captured at block 202. For example, processor 110 of computing device 106 can execute instructions 120 to infer cleaned IVUS images 124 from IVUS images 122 and ML model 126.

At block 208 “infer, based on the ML model and the plurality of images, a plurality of intermediate cleaned images” a plurality of intermediate cleaned images can be inferred from the plurality of images captured at block 202. For example, processor 110 of computing device 106 can execute instructions 120 to infer intermediate cleaned IVUS images (not shown) from IVUS images 122 and ML model 126.

Continuing from block 208 to decision block 210 “more ML models?” a determination of whether more ones of the multiple ML models are provided. For example, processor 110 of computing device 106 can execute instructions 120 to determine whether any more of ML models 126 remain to be used to generate an inference. As outlined above, multiple ML models 126 can be provided to remove noise artifacts (e.g., resulting from different types of noise, or the like). In such examples, the ML model 126 are executed serially to remove noise artifacts from the IVUS images 122. From decision block 210, routine 200 can continue to either block 212 or block 214. Routine 200 can continue from decision block 210 to block 212 based on a determination at decision block 210 that more ML models should be executed to remove noise artifacts, whereas routine 200 can continue from decision block 210 to block 214 based on a determination at decision block 210 that more ML models do not need to be executed to remove noise artifacts.

At block 212 “store the plurality of intermediate cleaned images as the plurality of images” the plurality of intermediate cleaned images can be stored (or designated as) the plurality of images and the routine can return to block 208 to generate a subsequent plurality of intermediate cleaned images from the prior intermediate cleaned images. For example, processor 110 of computing device 106 can execute instructions 120 to designate the previously inferred intermediate cleaned images as the plurality of images. After which, routine 200 can return to block 208 where a subsequent set of intermediate cleaned images are inferred from another one of the ML models 126.

At block 214 “store the plurality of intermediate cleaned images as the plurality of cleaned images” the plurality of intermediate cleaned images can be stored (or designated as) the plurality of cleaned images. For example, processor 110 of computing device 106 can execute instructions 120 to designate the previously inferred intermediate cleaned images as cleaned IVUS images 124.

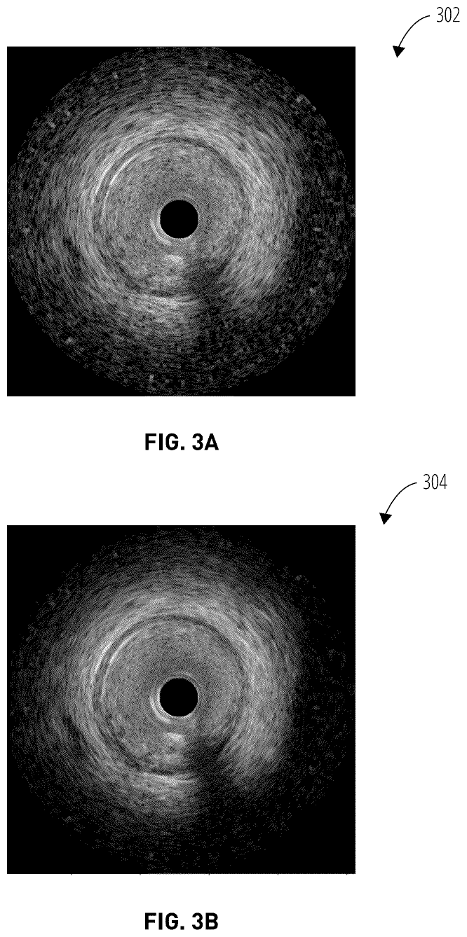

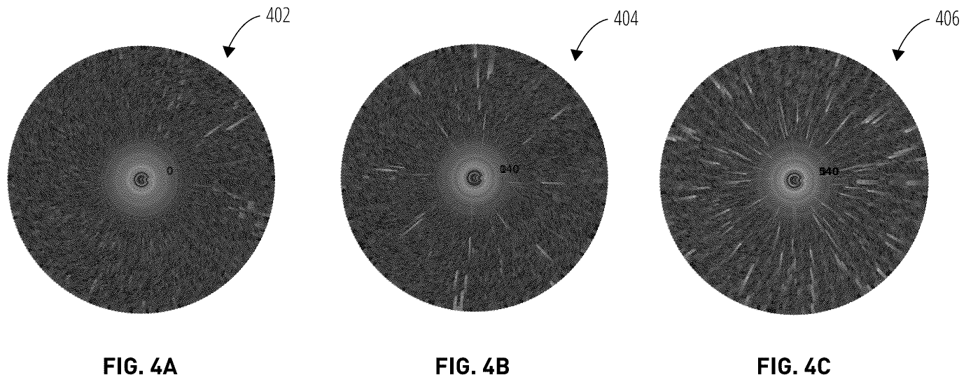

FIG. 3A and FIG. 3B illustrate an example of an IVUS image with blood speckle and other noise artifacts and the same IVUS image with the noise artifacts removed. For example, FIG. 3A illustrates IVUS image frame 302 which shows artifacts resulting from blood speckle noise. It is to be appreciated that blood flowing in the vessel exhibits a characteristic pattern of echogenicity, which inherently results is noise in IVUS images. This noise is manifest as textured echoes moving in a swirling pattern in the captured IVUS image frames. It is further to be appreciated that noise artifacts from blood speckle often increase as the operating frequency of the ultrasound increases. This leads to challenges in commercially implementing high definition IVUS transducers. The present disclosure can be provided to mitigate these challenges. IVUS image frame 302 depicted in FIG. 3A shows high levels of blood speckle noise artifacts, depicted as textured dots or grains in the image. The additional noise artifacts are especially easy to see around the outer circumference of the image.

Although there are conventional filters that can be applied to reduce or remove blood speckle noise artifacts, the present disclosure provides to train an ML model (e.g., ML model 126) to infer a cleaned IVUS image from an IVUS image having blood speckle noise artifacts. FIG. 3B illustrates cleaned IVUS image frame 304, which is IVUS image frame 302 after removing noise artifacts from blood speckle. As can be seen, cleaned IVUS image frame 304 shows significantly less grainy texture than does IVUS image frame 302.

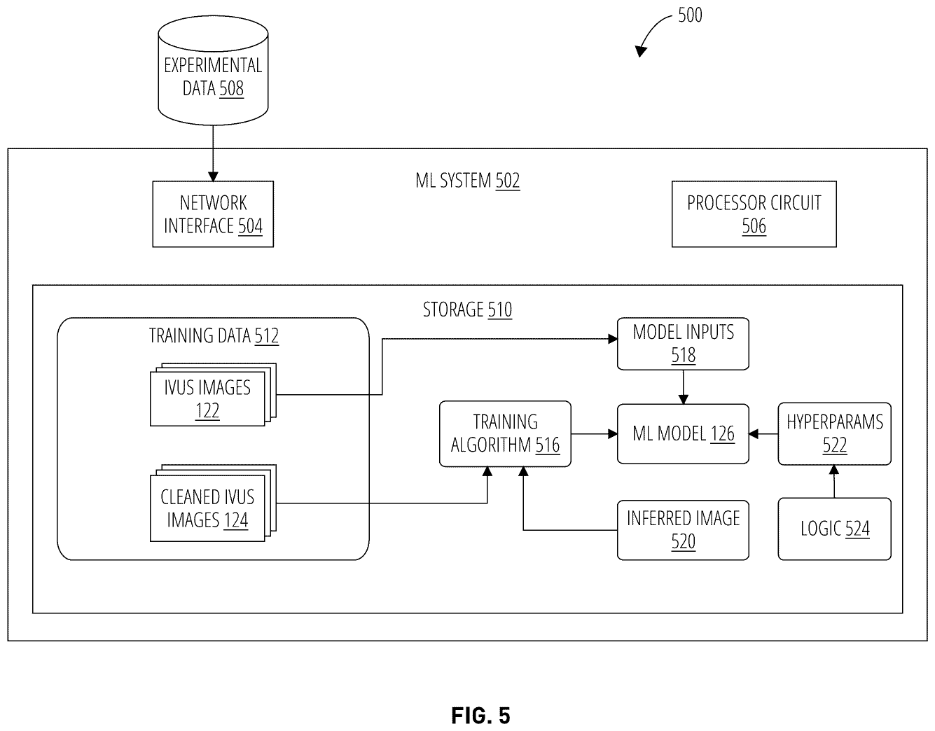

FIG. 4A, FIG. 4B, and FIG. 4C illustrates IVUS images containing another type of noise artifact. It will be appreciated that in addition to blood speckle noise, there may be outside sources of noise or interference. For example, a common cause of interference is power supplies from other medical equipment in the same lab. Another source of interference is radio frequency devices (e.g., cell phones, Bluetooth speakers, etc.) These other electronic devices are typically not synchronized with the IVUS system so generally they cause moving artifacts that look like a radial streaks in the image. In extreme cases these interference sources may cause artifacts that look like jagged circles around the center of the image. In some examples, the interference may be transient (e.g., appearing and then disappearing quickly). Ultimately, as more and more electronic devices saturate cardiology labs and equipment is upgraded with new and more technology, noise will increasingly be a barrier to high quality and consistent IVUS image quality. For example, FIG. 4A illustrates an IVUS image frame 402 captured in the absence of external electronic interference. As can be seen from this image, very minimal noise artifacts are depicted.

FIG. 4B illustrates an IVUS image frame 404 captured in the presence of another electronic system powered on, but the motor coupled to the system is not operating. As can be seen from this figure, IVUS image frame 404 depicts noise artifacts as radial streaks, likely from the interference caused by the other systems power supply.

FIG. 4C illustrates an IVUS image frame 406 captured in the presence of another electronic system powered on with a motor coupled to the system operating. As can be seen from this figure, IVUS image frame 406 depicts high levels of noise artifacts. For example, IVUS image frame 406 depicts significantly more radial streaks than IVUS image frame 404.

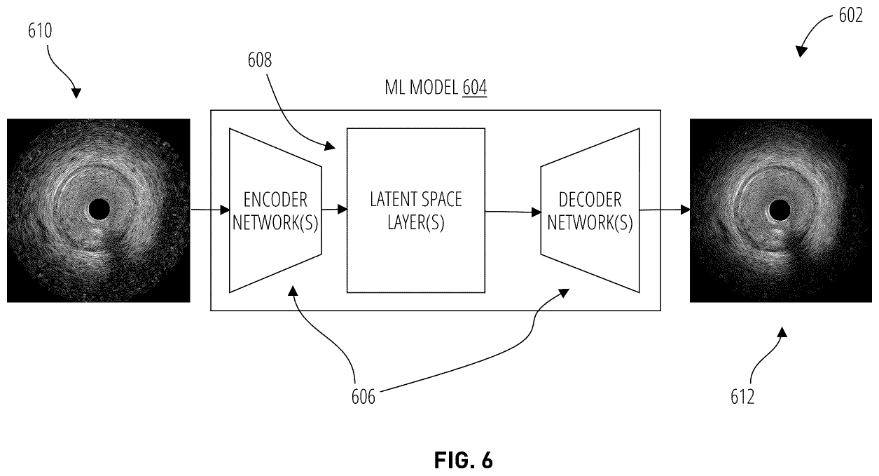

As noted, with some embodiments, processor 110 of computing device 106 can execute instructions 120 to generate cleaned IVUS images 124 using ML model 126. In such an example, the ML model 126 can be stored in memory 112 of computing device 106. It will be appreciated, that prior to being deployed, the ML model 126 is to be trained. FIG. 5 illustrates an ML environment 500, which can be used to train an ML model that may later be used to generate (or infer) cleaned IVUS images 124 as described herein.

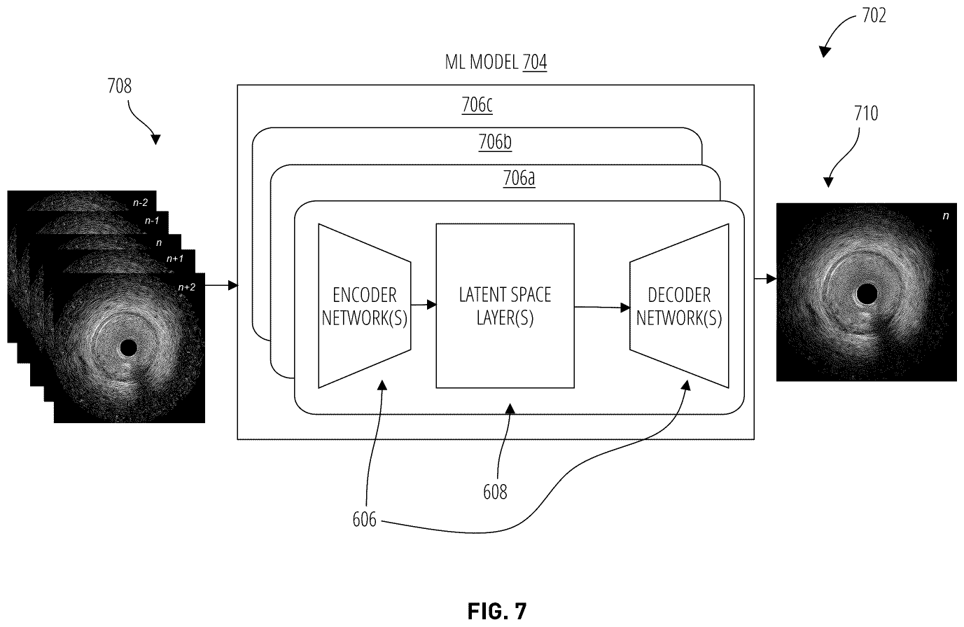

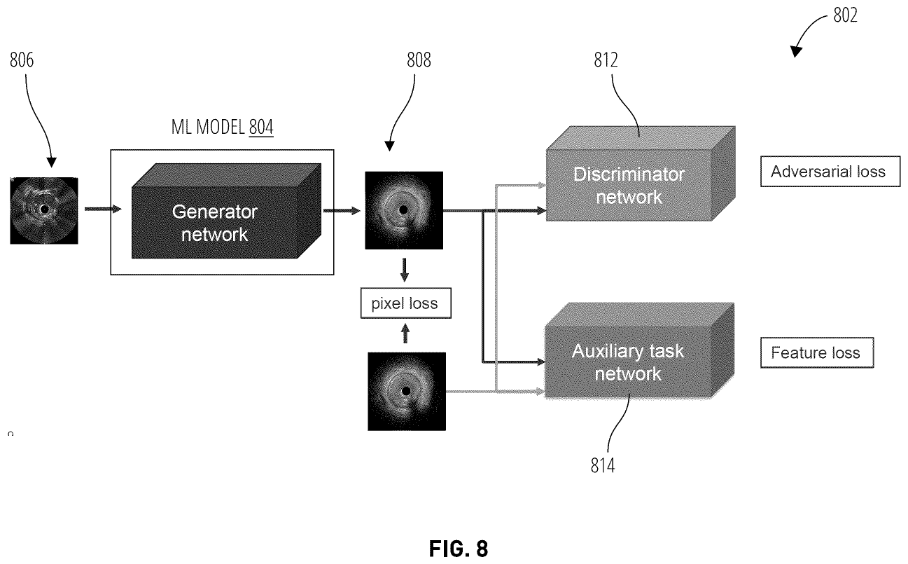

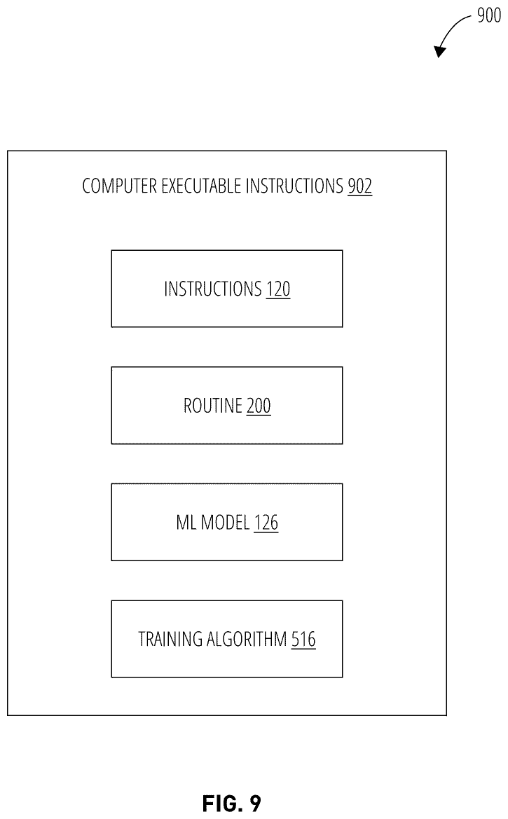

The ML environment 500 may include an ML system 502, such as a computing device that applies an ML algorithm to learn relationships. In this example, the ML algorithm can learn relationships between a set of inputs (e.g., IVUS images 122, or the like) and an output (e.g., cleaned IVUS images 124). This relationship is learned based on a training data 512, which can be collected from experimental data 508. The present disclosure contemplates several training architectures and configurations (e.g., see FIG. 6, FIG. 7, and FIG. 8) that correspond to both paired and unpaired images. Further it is contemplated that images with noise artifacts from different underlying noise sources will be included into a training dataset.

Experimental data 508 can include multiple IVUS images 122 and/or corresponding cleaned IVUS images 124 where the IVUS images 122 include noise artifacts. The experimental data 508 can be collected from actual IVUS images or can be synthetically generated from known noise patterns (e.g., using generative AI methods, or the like). Examples of this are explained in greater detail below. However, this figure depicts ML environment 500 configured to train ML model 126 where the training data 512 includes both original (e.g., noisy) images and corresponding clean images. In such an example, any decoder style network (e.g., NN, CNN, or the like) can be employed and trained using supervised learning techniques.

The experimental data 508 may be collocated with the ML system 502 (e.g., stored in a storage 510 of the ML system 502), may be remote from the ML system 502 and accessed via a network interface 504, or may be a combination of local and remote data.

As noted above, the ML system 502 may include a storage 510, which may include a hard drive, solid state storage, and/or random access memory. The storage 510 may hold training data 512. In general, training data 512 can include information elements or data structures comprising indications of IVUS images 122 and corresponding cleaned IVUS images 124.

The training data 512 may be applied to train ML model 126. As noted, depending on the application, different types of models may be used to form the basis of ML model 514. For instance, in the present example, an artificial neural network (ANN) may be particularly well-suited to learning associations between a raw IVUS image containing noise artifacts (e.g., IVUS images 122) and a cleaned IVUS images with the noise artifacts removed (e.g., cleaned IVUS images 124). Convoluted neural networks may also be well-suited to this task. Any suitable training algorithm 516 may be used to train the ML model 126. Nonetheless, the example depicted in FIG. 5 may be particularly well-suited to a supervised training algorithm or reinforcement learning training algorithm. For a supervised training algorithm, the ML system 502 may apply the IVUS images 122 as model inputs 518, to which an inferred image 520 is generated from the training data 512. The inferred image is compared to a ground truth image (e.g., cleaned IVUS images 124) and associations between the model inputs 518 (e.g., IVUS images 122) and the inferred image 520 (e.g., cleaned IVUS images 124) are learned. In a reinforcement learning scenario, training algorithm 516 may attempt to maximize some or all (or a weighted combination) of the model inputs 518 mappings to cleaned IVUS images 124 to produce ML model 126 having the least error. With some embodiments, training data 512 can be split into “training” and “testing” data wherein some subset of the training data 512 can be used to adjust the ML model 126 (e.g., internal weights of the model, or the like) while another, non-overlapping subset of the training data 512 can be used to measure an accuracy of the ML model 126 to infer (or generalize) a cleaned IVUS images 124 from “unseen” training data 512 (e.g., training data 512 not used to train ML model 126).

The ML model 126 may be applied using a processor circuit 506, which may include suitable hardware processing resources that operate on the logic and structures in the storage 510. The training algorithm 516 and/or the development of the trained ML model 126 may be at least partially dependent on hyperparameters 522. In exemplary embodiments, the model hyperparameters 522 may be automatically selected based on hyperparameter optimization logic 524, which may include any known hyperparameter optimization techniques as appropriate to the ML model 126 selected and the training algorithm 516 to be used. In optional, embodiments, the ML model 126 may be re-trained over time, to accommodate new knowledge and/or updated experimental data 508.

Once the ML model 126 is trained, it may be applied (e.g., by the processor circuit 506, by processor 110, or the like) to new input data (e.g., IVUS images 122 captured during a pre-PCI intervention, or the like). This input to the ML model 126 may be formatted according to a predefined model inputs 518 mirroring the way that the training data 512 was provided to the ML model 126. The ML model 126 may generate cleaned IVUS images 124 which may be, for example, a generalization or inference of the original or raw IVUS image (e.g., IVUS images 122) with noise artifacts removed.

The above description pertains to a particular kind of ML system 502, which applies supervised learning techniques given available training data with input/result pairs. However, the present invention is not limited to use with a specific ML paradigm, and other types of ML techniques may be used. For example, in some embodiments the ML system 502 may apply unsupervised learning algorithms, self-supervised learning algorithms, evolutionary algorithms, or other types of ML algorithms to train ML model 126 to generate cleaned IVUS images 124 from IVUS images 122.

FIG. 6 illustrates an example ML network architecture 602 that can be implemented to train ML model 604 using paired images. Once trained, ML model 604 could be implemented as ML model 126 or one of ML models 126. For example, images captured with blood speckle noise artifacts can be cleaned using de-speckle algorithms. As another example, noisy images and corresponding clean images can be generated using generative AI. Once the paired training dataset is generated, an ML model 604 comprising input and output layers 606 and latent layers 608. With some examples, the input and output layers 606 can be encoding and decoding networks, filters, or a combination of both. ML model 604 can be trained by minimizing the error between outputs generated from the noisy inputs and the corresponding ground truth outputs. For example, where the input 610 is the noisy input and the output 612 is the ground truth output, the ML model 604 can be trained by minimizing the function:

$$arg {min}_{θ} {\sum }_{i} L ( {f}_{θ} ( 610 ) , 612 ) ,$$

wherein L is the loss function and f is the training function.

FIG. 7 illustrates an example ML network architecture 702 that can be implemented to train ML model 704 using unpaired images. That is, ML model 704 can be trained using an unsupervised or self-supervised learning algorithm with noisy images that do not have corresponding clean images. Once trained, ML model 704 could be implemented as ML model 126 or one of ML models 126. In some embodiments, ML model 704 can be a CNN multiple sub-networks 706a, 706b, and 706c. Each one of sub-networks 706a, 706b, and 706c can include input and output layers 606 and latent layers 608. With some examples, the input and output layers 606 can be encoding and decoding networks, filters, convolution and deconvolution layers, bottleneck layers, or some combination of these.

Further, inputs 708 and output 710 are shown. ML model 704 can be implemented and trained where clean pairs cannot be generated from noisy samples. In such an example, the ML model 704 is configured to take multiple consecutive IVUS image frames (e.g., multiple ones of IVUS images 122) as input and output a clean image for a target frame from the multiple frames (e.g., typically the middle input frame). In this example, the input 708 includes frames x_{n-2:n+2} where the target frame is the middle frame n. The output 710 is a cleaned version of the target frame n.

It is noted that this approach will benefit from the temporal consistency of underlying morphological structures while noisy structures, like blood speckle, do not share the same temporal consistency. The ML model 704 will be trained using a training algorithm that penalizes the difference between the target frame and adjacent frames a loss function during training. ML model 704 may be implemented to target the removal of speckle noises (e.g., blood speckle, or the like).

FIG. 8 illustrates an example ML network architecture 802 that can be implemented to train ML model 804 using unpaired images. It is to be appreciated that several noise artifacts may manifest as image anomalies. In some embodiments, a training dataset can be generated by annotating noisy images and then a model can be trained using generative AI algorithms. For example, ML model 804 can be trained to generate clean images from annotated noisy images. Once trained, ML model 804 could be implemented as ML model 126 or one of ML models 126.

A variety of generative AI algorithms exist. ML network architecture 802 provides to train ML model 804 to generate output 808 from input 806 where the input 806 includes noise artifacts and the output is clean or has the noise artifacts removed. ML network architecture 802 provides to train ML model 804 using generative adversarial networks (GAN) or Generative Diffusion Models. To that end, ML network architecture 802 includes a discriminator network 812 and optionally, auxiliary task network 814. ML model 804 is trained to generate output 808 while discriminator network 812 is trained to detect whether output 808 is an authentic image or a generated image. ML model 804 and discriminator network 812 can be trained simultaneously in an unsupervised manner. Likewise, generation of output 808 can be improved by adding auxiliary task network 814. In some examples, auxiliary task network 814 can be trained to detect some features of the image and trained to maximize feature retention in the output 808.

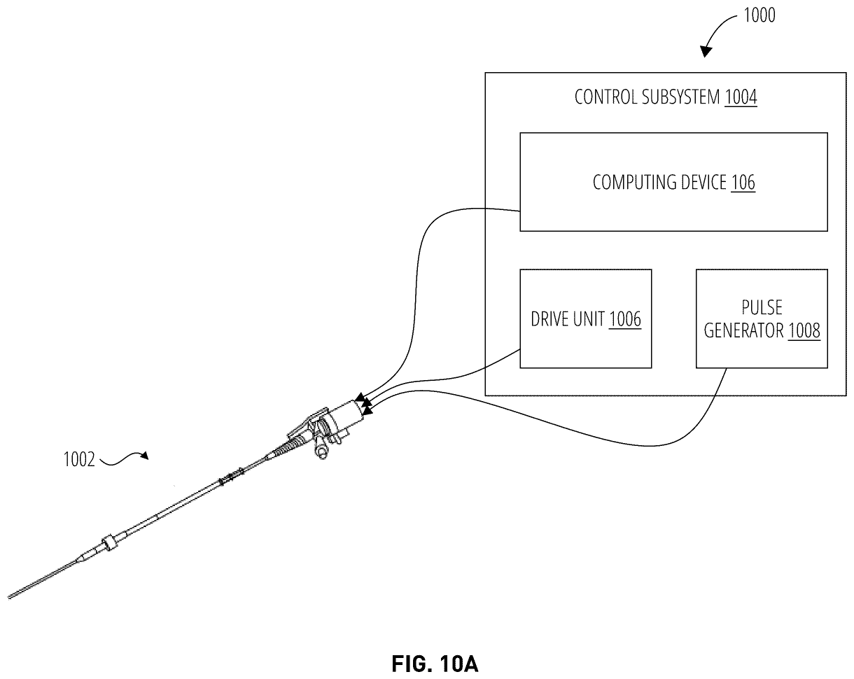

FIG. 9 illustrates computer-readable storage medium 900. Computer-readable storage medium 900 may comprise any non-transitory computer-readable storage medium or machine-readable storage medium, such as an optical, magnetic or semiconductor storage medium. In various embodiments, computer-readable storage medium 900 may comprise an article of manufacture. In some embodiments, computer-readable storage medium 900 may store computer executable instructions 902 with which circuitry (e.g., processor 110, or the like) can execute. For example, computer executable instructions 902 can include instructions to implement operations described with respect to routine 200, which can be specially programmed to cause noise artifact removal system 100 to perform the operations described with reference to routine 200 and FIG. 2. As another example, computer executable instructions 902 can include instructions 120, ML model 126, and/or training algorithm 516. Examples of computer-readable storage medium 900 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 902 may include any suitable type of code, such as source code, compiled code, interpreted code, executable code, static code, dynamic code, object-oriented code, visual code, and the like.

FIG. 10A, FIG. 10B, and FIG. 10C illustrate an example intravascular treatment system 1000 and are described together herein. FIG. 10A is a component level view while FIG. 10B and FIG. 10C are side and perspective views, respectively, of a portion of the intravascular treatment system 1000 of FIG. 10A. The intravascular treatment system 1000 takes the form of an IVUS imaging system and can be implemented as part of the noise artifact removal system 100 of FIG. 1. The intravascular treatment system 1000 includes a catheter 1002 and a control subsystem 1004. The control subsystem 1004 includes the computing device 106, a drive unit 1006 and a pulse generator 1008. The catheter 1002 and control subsystem 1004 are operably coupled, or more specifically, the catheter 1002 is electrically and/or mechanically coupled to the computing device 106, drive unit 1006, and pulse generator 1008 such that signals (e.g., control, measurement, image data, or the like) can be communicated between the catheter 1002 and control subsystem 1004.

It is noted that the computing device 106 includes display 116. However, in some applications, display 116 may be provided as a separate unit from computing device 106, for example, in a different housing, or the like. In some instances, the pulse generator 1008 forms electric pulses that may be input to one or more transducers 1030 disposed in the catheter 1002.

In some instances, mechanical energy from the drive unit 1006 may be used to drive an imaging core 1024 disposed in the catheter 1002. In some instances, electric signals transmitted from the one or more transducers 1030 may be input to the processor 110 of computing device 106 for processing as outlined here. For example, to be used to generate graphical information element 128. In some instances, the processed electric signals from the one or more transducers 1030 can also be displayed as one or more images on the display 116.

In some instances, the processor 110 may also be used to control the functioning of one or more of the other components of control subsystem 1004. For example, the processor 110 may be used to control at least one of the frequency or duration of the electrical pulses transmitted from the pulse generator 1008, the rotation rate of the imaging core 1024 by the drive unit 1006, the velocity or length of the pullback of the imaging core 1024 by the drive unit 1006, or one or more properties of one or more images formed on the display 116, such as, the graphical information element 128.

FIG. 10B is a side view of one embodiment of the catheter 1002 of the intravascular treatment system 1000 of FIG. 10A. The catheter 1002 includes an elongated member 1010 and a hub 1012. The elongated member 1010 includes a proximal end 1014 and a distal end 1016. In FIG. 10B, the proximal end 1014 of the elongated member 1010 is coupled to the catheter hub 1012 and the distal end 1016 of the elongated member 1010 is configured and arranged for percutaneous insertion into a patient. Optionally, the catheter 1002 may define at least one flush port, such as flush port 1018. The flush port 1018 may be defined in the hub 1012. The hub 1012 may be configured and arranged to couple to the control subsystem 1004 of intravascular treatment system 1000. In some instances, the elongated member 1010 and the hub 1012 are formed as a unitary body. In other instances, the elongated member 1010 and the catheter hub 1012 are formed separately and subsequently assembled.

FIG. 10C is a perspective view of one embodiment of the distal end 1016 of the elongated member 1010 of the catheter 1002. The elongated member 1010 includes a sheath 1020 with a longitudinal axis (e.g., a central longitudinal axis extending axially through the center of the sheath 1020 and/or the catheter 1002) and a lumen 1022. An imaging core 1024 is disposed in the lumen 1022. The imaging core 1024 includes an imaging device 1026 coupled to a distal end of a driveshaft 1028 that is rotatable either manually or using a computer-controlled drive mechanism. One or more transducers 1030 may be mounted to the imaging device 1026 and employed to transmit and receive acoustic signals. The sheath 1020 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

In some instances, for example as shown in these figures, an array of transducers 1030 are mounted to the imaging device 1026. Alternatively, a single transducer may be employed. Any suitable number of transducers 1030 can be used. For example, there can be two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used. When a plurality of transducers 1030 are employed, the transducers 1030 can be configured into any suitable arrangement including, for example, an annular arrangement, a rectangular arrangement, or the like.

The one or more transducers 1030 may be formed from materials capable of transforming applied electrical pulses to pressure distortions on the surface of the one or more transducers 1030, and vice versa. Examples of suitable materials include piezoelectric ceramic materials, piezocomposite materials, piezoelectric plastics, barium titanates, lead zirconate titanates, lead metaniobates, polyvinylidene fluorides, and the like. Other transducer technologies include composite materials, single-crystal composites, and semiconductor devices (e.g., capacitive micromachined ultrasound transducers (“cMUT”), piezoelectric micromachined ultrasound transducers (“pMUT”), or the like).

The pressure distortions on the surface of the one or more transducers 1030 form acoustic pulses of a frequency based on the resonant frequencies of the one or more transducers 1030. The resonant frequencies of the one or more transducers 1030 may be affected by the size, shape, and material used to form the one or more transducers 1030. The one or more transducers 1030 may be formed in any shape suitable for positioning within the catheter 1002 and for propagating acoustic pulses of a desired frequency in one or more selected directions. For example, transducers may be disc-shaped, block-shaped, rectangular-shaped, oval-shaped, and the like. The one or more transducers may be formed in the desired shape by any process including, for example, dicing, dice and fill, machining, microfabrication, and the like.

As an example, each of the one or more transducers 1030 may include a layer of piezoelectric material sandwiched between a matching layer and a conductive backing material formed from an acoustically absorbent material (e.g., an epoxy substrate with tungsten particles). During operation, the piezoelectric layer may be electrically excited to cause the emission of acoustic pulses.

The one or more transducers 1030 can be used to form a radial cross-sectional image of a surrounding space. Thus, for example, when the one or more transducers 1030 are disposed in the catheter 1002 and inserted into a blood vessel of a patient, the one more transducers 1030 may be used to form an image of the walls of the blood vessel and tissue surrounding the blood vessel. The imaging core 1024 and one or more transducers produce IVUS images 122.

The imaging core 1024 is rotated about the longitudinal axis of the catheter 1002. As the imaging core 1024 rotates, the one or more transducers 1030 emit acoustic signals in different radial directions (e.g., along different radial scan lines). For example, the one or more transducers 1030 can emit acoustic signals at regular (or irregular) increments, such as 256 radial scan lines per revolution, or the like. It will be understood that other numbers of radial scan lines can be emitted per revolution, instead.

When an emitted acoustic pulse with sufficient energy encounters one or more medium boundaries, such as one or more tissue boundaries, a portion of the emitted acoustic pulse is reflected to the emitting transducer as an echo pulse. Each echo pulse that reaches a transducer with sufficient energy to be detected is transformed to an electrical signal in the receiving transducer. The one or more transformed electrical signals are transmitted to the processor 110 of the computing device 106 where it is processed to form cleaned IVUS images 124 and subsequently generate graphical information element 128 to be displayed on display 116. In some instances, the rotation of the imaging core 1024 is driven by the drive unit 1006, which can be disposed in control subsystem 1004. In alternate embodiments, the one or more transducers 1030 are fixed in place and do not rotate. In which case, the driveshaft 1028 may, instead, rotate a mirror that reflects acoustic signals to and from the fixed one or more transducers 1030.

When the one or more transducers 1030 are rotated about the longitudinal axis of the catheter 1002 emitting acoustic pulses, a plurality of images can be formed that collectively form a radial cross-sectional image (e.g., a tomographic image) of a portion of the region surrounding the one or more transducers 1030, such as the walls of a blood vessel of interest and tissue surrounding the blood vessel. The radial cross-sectional image can form the basis of IVUS images 122 and can optionally be displayed on display 116. The at least one of the imaging core 1024 can be either manually rotated or rotated using a computer-controlled mechanism.

The imaging core 1024 may also move longitudinally along the blood vessel within which the catheter 1002 is inserted so that a plurality of cross-sectional images may be formed along a longitudinal length of the blood vessel. During an imaging procedure the one or more transducers 1030 may be retracted (e.g., pulled back) along the longitudinal length of the catheter 1002. The catheter 1002 can include at least one telescoping section that can be retracted during pullback of the one or more transducers 1030. In some instances, the drive unit 1006 drives the pullback of the imaging core 1024 within the catheter 1002. The drive unit 1006 pullback distance of the imaging core can be any suitable distance including, for example, at least 5 cm, 10 cm, 15 cm, 20 cm, 25 cm, or more. The entire catheter 1002 can be retracted during an imaging procedure either with or without the imaging core 1024 moving longitudinally independently of the catheter 1002.

A stepper motor may, optionally, be used to pull back the imaging core 1024. The stepper motor can pull back the imaging core 1024 a short distance and stop long enough for the one or more transducers 1030 to capture an image or series of images before pulling back the imaging core 1024 another short distance and again capturing another image or series of images, and so on.

The quality of an image produced at different depths from the one or more transducers 1030 may be affected by one or more factors including, for example, bandwidth, transducer focus, beam pattern, as well as the frequency of the acoustic pulse. The frequency of the acoustic pulse output from the one or more transducers 1030 may also affect the penetration depth of the acoustic pulse output from the one or more transducers 1030. In general, as the frequency of an acoustic pulse is lowered, the depth of the penetration of the acoustic pulse within patient tissue increases. In some instances, the intravascular treatment system 1000 operates within a frequency range of 5 MHz to 1000 MHz.

One or more conductors 1032 can electrically couple the transducers 1030 to the control subsystem 1004. In which case, the one or more conductors 1032 may extend along a longitudinal length of the rotatable driveshaft 1028.

The catheter 1002 with one or more transducers 1030 mounted to the distal end 1016 of the imaging core 1024 may be inserted percutaneously into a patient via an accessible blood vessel, such as the femoral artery, femoral vein, or jugular vein, at a site remote from the selected portion of the selected region, such as a blood vessel, to be imaged. The catheter 1002 may then be advanced through the blood vessels of the patient to the selected imaging site, such as a portion of a selected blood vessel.

An image or image frame (“frame”) can be generated each time one or more acoustic signals are output to surrounding tissue and one or more corresponding echo signals are received by the imaging device 1026 and transmitted to the processor 110 of the computing device 106. Alternatively, an image or image frame can be a composite of scan lines from a full or partial rotation of the imaging core or device. A plurality (e.g., a sequence) of frames may be acquired over time during any type of movement of the imaging device 1026. For example, the frames can be acquired during rotation and pullback of the imaging device 1026 along the target imaging location. It will be understood that frames may be acquired both with or without rotation and with or without pullback of the imaging device 1026. Moreover, it will be understood that frames may be acquired using other types of movement procedures in addition to, or in lieu of, at least one of rotation or pullback of the imaging device 1026.

In some instances, when pullback is performed, the pullback may be at a constant rate, thus providing a tool for potential applications able to compute longitudinal vessel/plaque measurements. In some instances, the one or more acoustic signals are output to surrounding tissue at constant intervals of time. In some instances, the one or more corresponding echo signals are received by the imaging device 1026 and transmitted to the processor 110 of the computing device 106 at constant intervals of time. In some instances, the resulting frames are generated at constant intervals of time.

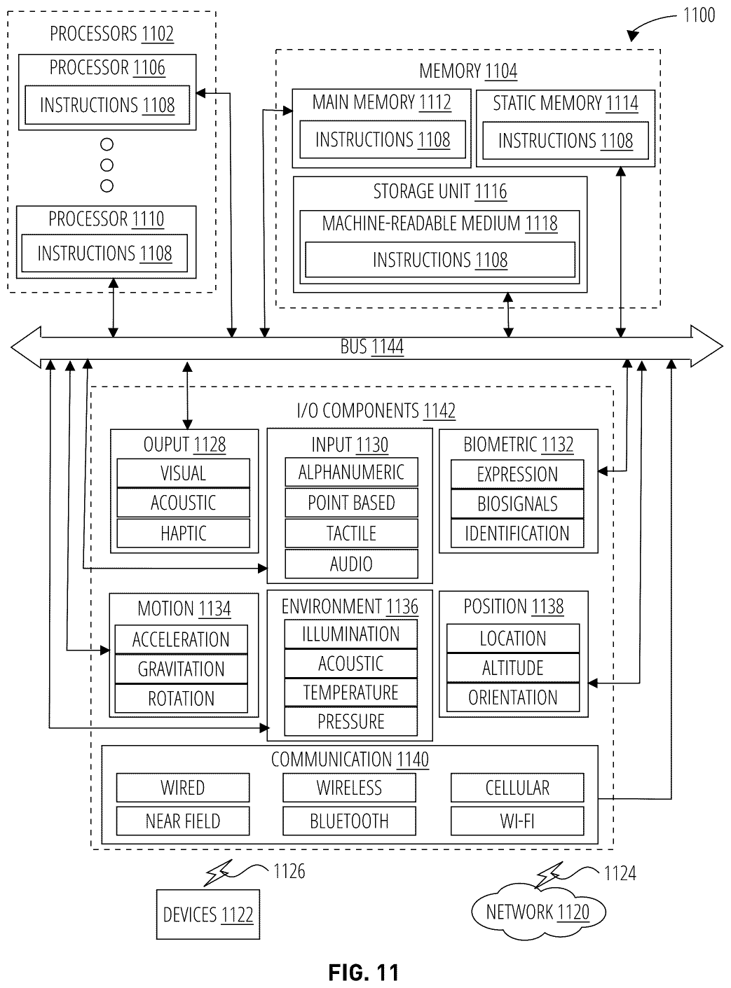

FIG. 11 illustrates a diagrammatic representation of a machine 1100 in the form of a computer system within which a set of instructions may be executed for causing the machine to perform any one or more of the methodologies discussed herein. More specifically, FIG. 11 shows a diagrammatic representation of the machine 1100 in the example form of a computer system, within which instructions 1108 (e.g., software, a program, an application, an applet, an app, or other executable code) for causing the machine 1100 to perform any one or more of the methodologies discussed herein may be executed. For example, the instructions 1108 may cause the machine 1100 to execute instructions 120, routine 200 of FIG. 2, training algorithm 516, or the like. More generally, the instructions 1108 may cause the machine 1100 to train ML models (e.g., ML model 126) to infer clean IVUS images from noisy IVUS images and/or to generate clean IVUS images from noisy IVUS images captured during a PCI procedure as described herein.

The instructions 1108 transform the general, non-programmed machine 1100 into a particular machine 1100 programmed to carry out the described and illustrated functions in a specific manner. In alternative embodiments, the machine 1100 operates as a standalone device or may be coupled (e.g., networked) to other machines. In a networked deployment, the machine 1100 may operate in the capacity of a server machine or a client machine in a server-client network environment, or as a peer machine in a peer-to-peer (or distributed) network environment. The machine 1100 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1108, sequentially or otherwise, that specify actions to be taken by the machine 1100. Further, while only a single machine 1100 is illustrated, the term “machine” shall also be taken to include a collection of machines 200 that individually or jointly execute the instructions 1108 to perform any one or more of the methodologies discussed herein.

The machine 1100 may include processors 1102, memory 1104, and I/O components 1142, which may be configured to communicate with each other such as via a bus 1144. In an example embodiment, the processors 1102 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1106 and a processor 1110 that may execute the instructions 1108. The term “processor” is intended to include multi-core processors that may comprise two or more independent processors (sometimes referred to as “cores”) that may execute instructions contemporaneously. Although FIG. 11 shows multiple processors 1102, the machine 1100 may include a single processor with a single core, a single processor with multiple cores (e.g., a multi-core processor), multiple processors with a single core, multiple processors with multiples cores, or any combination thereof.

The memory 1104 may include a main memory 1112, a static memory 1114, and a storage unit 1116, both accessible to the processors 1102 such as via the bus 1144. The main memory 1104, the static memory 1114, and storage unit 1116 store the instructions 1108 embodying any one or more of the methodologies or functions described herein. The instructions 1108 may also reside, completely or partially, within the main memory 1112, within the static memory 1114, within machine-readable medium 1118 within the storage unit 1116, within at least one of the processors 1102 (e.g., within the processor's cache memory), or any suitable combination thereof, during execution thereof by the machine 1100.

The I/O components 1142 may include a wide variety of components to receive input, provide output, produce output, transmit information, exchange information, capture measurements, and so on. The specific I/O components 1142 that are included in a particular machine will depend on the type of machine. For example, portable machines such as mobile phones will likely include a touch input device or other such input mechanisms, while a headless server machine will likely not include such a touch input device. It will be appreciated that the I/O components 1142 may include many other components that are not shown in FIG. 11. The I/O components 1142 are grouped according to functionality merely for simplifying the following discussion and the grouping is in no way limiting. In various example embodiments, the I/O components 1142 may include output components 1128 and input components 1130. The output components 1128 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1130 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1142 may include biometric components 1132, motion components 1134, environmental components 1136, or position components 1138, among a wide array of other components. For example, the biometric components 1132 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1134 may include acceleration sensor components (e.g., accelerometer), gravitation sensor components, rotation sensor components (e.g., gyroscope), and so forth. The environmental components 1136 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1138 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1142 may include communication components 1140 operable to couple the machine 1100 to a network 1120 or devices 1122 via a coupling 1124 and a coupling 1126, respectively. For example, the communication components 1140 may include a network interface component or another suitable device to interface with the network 1120. In further examples, the communication components 1140 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1122 may be another machine or any of a wide variety of peripheral devices (e.g., a peripheral device coupled via a USB).

Moreover, the communication components 1140 may detect identifiers or include components operable to detect identifiers. For example, the communication components 1140 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1140, such as location via Internet Protocol (IP) geolocation, location via Wi-Fi® signal triangulation, location via detecting an NFC beacon signal that may indicate a particular location, and so forth.

The various memories (i.e., memory 1104, main memory 1112, static memory 1114, and/or memory of the processors 1102) and/or storage unit 1116 may store one or more sets of instructions and data structures (e.g., software) embodying or utilized by any one or more of the methodologies or functions described herein. These instructions (e.g., the instructions 1108), when executed by processors 1102, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1120 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1120 or a portion of the network 1120 may include a wireless or cellular network, and the coupling 1124 may be a Code Division Multiple Access (CDMA) connection, a Global System for Mobile communications (GSM) connection, or another type of cellular or wireless coupling. In this example, the coupling 1124 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1108 may be transmitted or received over the network 1120 using a transmission medium via a network interface device (e.g., a network interface component included in the communication components 1140) and utilizing any one of several well-known transfer protocols (e.g., hypertext transfer protocol (HTTP)). Similarly, the instructions 1108 may be transmitted or received using a transmission medium via the coupling 1126 (e.g., a peer-to-peer coupling) to the devices 1122. The terms “transmission medium” and “signal medium” mean the same thing and may be used interchangeably in this disclosure. The terms “transmission medium” and “signal medium” shall be taken to include any intangible medium that can store, encoding, or carrying the instructions 1108 for execution by the machine 1100, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A computing device to couple to an intravascular imaging device, the computing device comprising:
a processor;
an interconnect coupled to the processor, the interconnect configured to couple to an intravascular imaging device; and
a memory device, the memory device comprising instructions that when executed by the processor cause the processor to:
receive, from the intravascular imaging device, a plurality of images associated with a vessel of a patient, the plurality of images comprising multidimensional and multivariate images; and
infer, using a machine learning (ML) model, a plurality of cleaned images corresponding to the plurality of images, wherein the plurality of images comprises one or more noisy artifacts and wherein at least one of the one or more noisy artifacts is removed from the plurality of cleaned images.

2. The computing device of claim 1, the instructions when executed further cause the processor to:
generate a graphical information element comprising an indication of the plurality of cleaned images; and
cause the graphical information element to be displayed on a display coupled to the computing device.

3. The computing device of claim 1, wherein the plurality of images are intravascular ultrasound (IVUS) images.

4. The computing device of claim 1, wherein the ML model comprises a plurality of ML models and wherein inferring the plurality of cleaned images comprises generating an inference from the plurality of ML models in serial.

5. The computing device of claim 1, wherein the ML model comprises a first ML model and a second ML model and wherein inferring the plurality of cleaned images comprises:
inferring, by the computing device using the first ML model with the plurality of images as input, a plurality of intermediate cleaned images corresponding to the plurality of images; and
inferring, by the computing device using the second ML model with the plurality of intermediate cleaned images as input, the plurality of cleaned images corresponding to the plurality of images.

6. The computing device of claim 1, wherein the ML model comprises a neural network (NN) or a convoluted neural network (CNN) architecture.

7. The computing device of claim 1, wherein the ML model is trained using a supervised training algorithm with a training data set that comprises a plurality of images comprising one or more noisy artifacts and, for each of the plurality of images, a ground truth image that does not comprise the one or more noisy artifacts.

8. The computing device of claim 1, wherein the ML model is trained using an unsupervised training algorithm with a training data set that comprises a plurality of images comprising one or more noisy artifacts and wherein the network is configured to receive at least two or more of the plurality of images as input and generate a cleaned image corresponding to a one of the at least two or more of the plurality of images, wherein the cleaned image does not comprise the one or more noisy artifacts.

9. One or more computer readable storage devices comprising instructions, which when executed by a processor of a computing device configured to couple to an intravascular imaging device, cause the computing device to:
receive, from the intravascular imaging device, a plurality of images associated with a vessel of a patient, the plurality of images comprising multidimensional and multivariate images; and
infer, using a machine learning (ML) model, a plurality of cleaned images corresponding to the plurality of images, wherein the plurality of images comprises one or more noisy artifacts and wherein at least one of the one or more noisy artifacts is removed from the plurality of cleaned images.

10. The one or more computer readable storage devices of claim 9, wherein the plurality of images are intravascular ultrasound (IVUS) images.

11. The one or more computer readable storage devices of claim 9, wherein the one or more noisy artifacts correspond to blood speckle noise, electromagnetic interference, and/or radio frequency interference.

12. The one or more computer readable storage devices of claim 9, wherein the ML model comprises a plurality of ML models and wherein inferring the plurality of cleaned images comprises generating an inference from the plurality of ML models in serial.

13. The one or more computer readable storage devices of claim 9, wherein the ML model comprises a first ML model and a second ML model and wherein inferring the plurality of cleaned images comprises:
inferring, by the computing device using the first ML model with the plurality of images as input, a plurality of intermediate cleaned images corresponding to the plurality of images; and
inferring, by the computing device using the second ML model with the plurality of intermediate cleaned images as input, the plurality of cleaned images corresponding to the plurality of images.

14. The one or more computer readable storage devices of claim 9, wherein the ML model is trained using a Diffusion Network training algorithm iteratively optimizing the network parameters.

15. The one or more computer readable storage devices of claim 9, wherein the ML model is trained using a generative adversarial network (GAN) training algorithm comprising a discriminator network.

16. The one or more computer readable storage devices of claim 15, wherein the GAN training algorithm comprises an auxiliary task network.

17. A method for removing noisy artifacts from a series of intravascular images, comprising:
receiving, at the computing device from an intravascular imaging device, a plurality of images associated with a vessel of the patient, the plurality of images comprising multidimensional and multivariate images; and
inferring, by the computing device using a machine learning (ML) model, a plurality of cleaned images corresponding to the plurality of images, wherein the plurality of images comprise one or more noisy artifacts and wherein at least one of the one or more noisy artifacts is removed from the plurality of cleaned images.

18. The method of claim 2, wherein the plurality of images are intravascular ultrasound (IVUS) images.

19. The method of claim 2, wherein the ML model comprises a plurality of ML models and wherein inferring the plurality of cleaned images comprises generating an inference from the plurality of ML models in serial.

20. The method of claim 2, wherein the ML model comprises a first ML model and a second ML model and wherein inferring the plurality of cleaned images comprises:
inferring, by the computing device using the first ML model with the plurality of images as input, a plurality of intermediate cleaned images corresponding to the plurality of images; and
inferring, by the computing device using the second ML model with the plurality of intermediate cleaned images as input, the plurality of cleaned images corresponding to the plurality of images.
